(19) United States
(12) Patent Application Publication
(10) Pub. No.: US 2024/0420816 A1
(43) Pub. Date: Dec. 19, 2024

(54) METHODS AND SYSTEMS OF FACILITATING MANAGING MEDICATION FOR A PATIENT

(71) Applicant: Tyler Lee Edwards, Archie, MO (US)

(72) Inventor: Tyler Lee Edwards, Archie, MO (US)

(21) Appl. No.: 18/453,786

(22) Filed: Aug. 22, 2023

Related U.S. Application Data
(60) Provisional application No. 63/508,659, filed on Jun. 16, 2023.

(51) Int. Cl. G16H 20/10; G16H 10/60; G16H 40/40; G16H 70/40

(52) U.S. Cl. CPC G16H 20/10; G16H 10/60; G16H 40/40; G16H 70/40

(57) ABSTRACT

Disclosed herein is a method of facilitating managing medication for a patient. Accordingly, the method may include receiving a prescription information from a patient device, receiving a medication information corresponding to at least one medication from the patient device, analyzing the medication information and the prescription information, generating a medication consumption information for consuming the medication, obtaining a current timing information associated with a current time of the patient, analyzing the current timing information and the medication consumption information, determining a match of the current timing information and the medication consumption information, selecting a first medication from the at least one medication, generating a notification based on the selecting, and transmitting the notification to the patient device.

### FIELD OF THE INVENTION

Generally, the present disclosure relates to the field of data processing. More specifically, the present disclosure relates to methods and systems of facilitating managing medication for a patient.

### BACKGROUND OF THE INVENTION

The field of data processing is technologically important to several industries, business organizations, and/or individuals. In particular, the use of data processing is prevalent for facilitating managing medication for patients.

Existing techniques for managing medication for patients are deficient with regard to several aspects. For instance, outpatients do not get continuous medical surveillance from medical staff, unlike inpatients. The absence of attention from medical staff may cause human error during the treatment of outpatients. Sometimes patients are overwhelmed by a medical event that they themselves and even their caretakers may not be in the right mindset to handle medication procedures.

Therefore, there is a need for improved methods and systems for facilitating managing medications that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a method of facilitating managing medication for a patient, in accordance with some embodiments. Accordingly, the method may include receiving, using a communication device, a prescription information associated with the patient from at least one patient device associated with the patient. Further, the method may include receiving, using the communication device, at least one medication information corresponding to at least one medication to be consumed by the patient from the at least one patient device. Further, the method may include analyzing, using a processing device, the at least one medication information and the prescription information. Further, the method may include generating, using the processing device, a medication consumption information for consuming the at least one medication by the patient based on the analyzing. Further, the medication consumption information may include at least one medication identifier, at least one timing, and at least one dosage associated with the at least one medication. Further, the method may include obtaining, using the processing device, a current timing information associated with a current time of the patient. Further, the method may include analyzing, using the processing device, the current timing information and the medication consumption information. Further, the method may include determining, using the processing device, a match of the current timing information and the medication consumption information. Further, the method may include selecting, using the processing device, at least one first medication from the at least one medication based on the determining of the match. Further, at least one first medication may be to be consumed by the patient at the current time. Further, the method may include generating, using the processing device, at least one notification based on the selecting of the at least one first medication. Further, the at least one notification may include at least one first medication consumption information from the medication consumption information. Further, the at least one first medication consumption information may be associated with the at least one first medication. Further, the method may include transmitting, using the communication device, the at least one notification to the at least one patient device.

Further disclosed herein is a system of facilitating managing medication for a patient, in accordance with some embodiments. Accordingly, the system may include a communication device configured for receiving a prescription information associated with the patient from at least one patient device associated with the patient. Further, the communication device may be configured for receiving at least one medication information corresponding to at least one medication to be consumed by the patient from the at least one patient device. Further, the communication device may be configured for transmitting at least one notification to the at least one patient device. Further, the system may include a processing device communicatively coupled with the communication device. Further, the processing device may be configured for analyzing the at least one medication information and the prescription information. Further, the processing device may be configured for generating a medication consumption information for consuming the at least one medication by the patient based on the analyzing. Further, the medication consumption information may include at least one medication identifier, at least one timing, and at least one dosage associated with the at least one medication. Further, the processing device may be configured for obtaining a current timing information associated with a current time of the patient. Further, the processing device may be configured for analyzing the current timing information and the medication consumption information. Further, the processing device may be configured for determining a match of the current timing information and the medication consumption information. Further, the processing device may be configured for selecting at least one first medication from the at least one medication based on the determining of the match. Further, at least one first medication may be to be consumed by the patient at the current time. Further, the processing device may be configured for generating the at least one notification based on the selecting of the at least one first medication. Further, the at least one notification may include at least one first medication consumption information from the medication consumption information. Further, the at least one first medication consumption information may be associated with the at least one first medication.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a flow chart of a method 200 of facilitating managing medication for a patient, in accordance with some embodiments.

FIG. 3 is a flow chart of a method 300 of facilitating managing medication for the patient, in accordance with some embodiments.

FIG. 4 is a flow chart of a method 400 of facilitating managing medication for the patient, in accordance with some embodiments.

FIG. 5 is a flow chart of a method 500 of facilitating managing medication for the patient, in accordance with some embodiments.

FIG. 6 is a flow chart of a method 600 of facilitating managing medication for the patient, in accordance with some embodiments.

FIG. 7 is a flow chart of a method 700 of facilitating managing medication for the patient, in accordance with some embodiments.

FIG. 8 is a flow chart of a method 800 of facilitating managing medication for the patient, in accordance with some embodiments.

FIG. 9 is a flow chart of a method 900 of facilitating managing medication for the patient, in accordance with some embodiments.

FIG. 10 is a block diagram of a system 1000 of facilitating managing medication for a patient, in accordance with some embodiments.

FIG. 11 is a block diagram of the system 1000 of facilitating managing medication for the patient, in accordance with some embodiments.

FIG. 12 is a block diagram of the system 1000 of facilitating managing medication for the patient, in accordance with some embodiments.

FIG. 13 is a block diagram of the system 1000 of facilitating managing medication for the patient, in accordance with some embodiments.

FIG. 14 is a block diagram of the system 1000 of facilitating managing medication for the patient, in accordance with some embodiments.

FIG. 15 is a flowchart of a method 1500 for facilitating medical assistance to patients, in accordance with some embodiments.

FIG. 16 is a flowchart of a method 1600 for facilitating medical assistance to patients, in accordance with some embodiments.

FIG. 17 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods and systems of facilitating managing medication for a patient, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosure describes methods and systems of facilitating managing medication for a patient. Further, the disclosed system may be configured for assisting a patient such as an outpatient sent home after a hospital visit with instructions to pick up medications at a local pharmacy.

Further, in an instance, a patient who has recently had surgery has been released from the hospital and sent home for recovery. The patient needs to pick up five medications from his local pharmacy. The patient picks up his local pharmacy prescriptions and confirms with the pharmacy the outpatient medication schedule he needs to adhere to. The patient has his personal Electronic Health Record (EHR) downloaded on a smartphone. Opening up the camera on the smartphone, the patient may simply scan the QR code on his medication and it may ask for two-factor authentication. This is a HIPAA requirement in order to protect patients. Once the two-factor authentication is achieved, the patient's mobile version of their health record appears with the correct MRN.

Barcode scanning has been proven to improve patient safety, and this may be a safety checkpoint to make sure it is the correct medication that the provider himself orders.

The patient may scan all five of the medications. Further, an application associated with the disclosed system then gives the patient the option to choose what his awake schedule is i.e. 9-5, night shift, hourly. Once the patient selects their awake schedule that suits their lifestyle, the disclosed system may use an algorithm for processing all the medication instructions per the pharmacist and starts creating medication alarms from their EHR to alert the patient when to take their medication. This is a reduction in human error. The alert may then give the patient three options to choose from, along the lines of ‘Snooze, Done, Dismiss’.

Once the patient selects ‘Done’ after taking his medication, that becomes a reportable event. Using the Home Medication Metric, hospital organizations may finally get a glimpse into the second leading cause of hospital readmissions home medication compliance. Simple things that healthcare organizations may implement via mobile messaging to help increase home medication compliance and decrease readmission rates.

Further, the disclosed system may be associated with a software platform or the app. Further, the patient has MyChart app, an exemplary embodiment of the disclosed software platform, on a smartphone associated with the patient. Further, the patient has the MyChart app installed on the smartphone or a mobile device and opens the camera within the app. The camera within the app (HIPPA compliant) then pulls up. The patient may then scan a QR code on the medication associated with the patient's MRN. Further, all the medications may be sent to the outpatient pharmacy to a CVS in this instance. Further, after being discharged from the hospital, the physician prescribes 4 medications that the patient needs to be taking at home. Once the patient scans all the medication QR codes, the algorithm then creates a series of alarms based on the pharmacist's instructions. Increased the patient safety by removing the human error element. Sometimes people are so overwhelmed by a medical event that the patient or their caretakers are not in the right mindset to be doing the math to see when the patient needs to take their medicines. The algorithm may do it for the patients ensuring the safety of the patient.

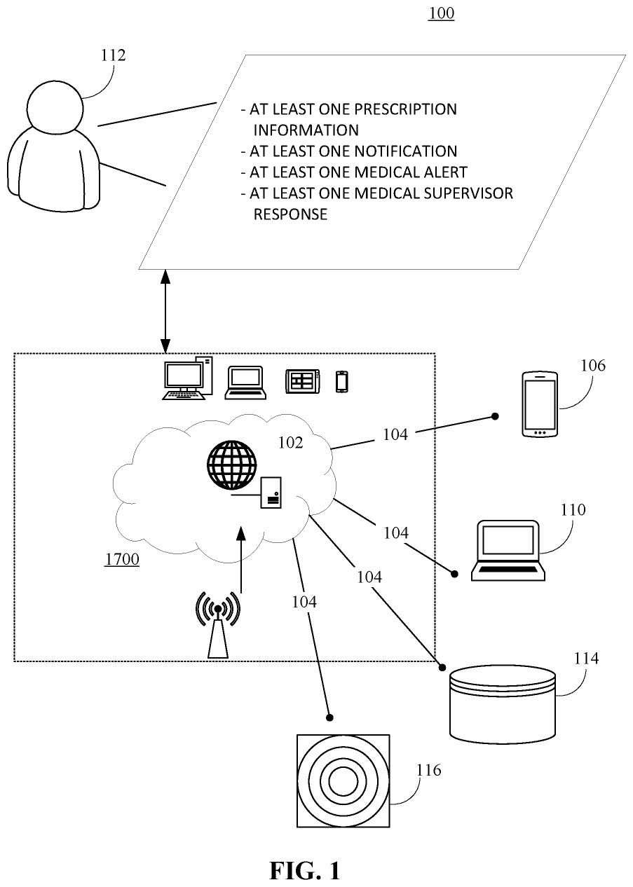

Referring now to figures, FIG. 1 is an illustration of an online platform 100 consistent with various embodiments of the present disclosure. By way of non-limiting example, the online platform 100 to facilitate managing medication for a patient may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 1700.

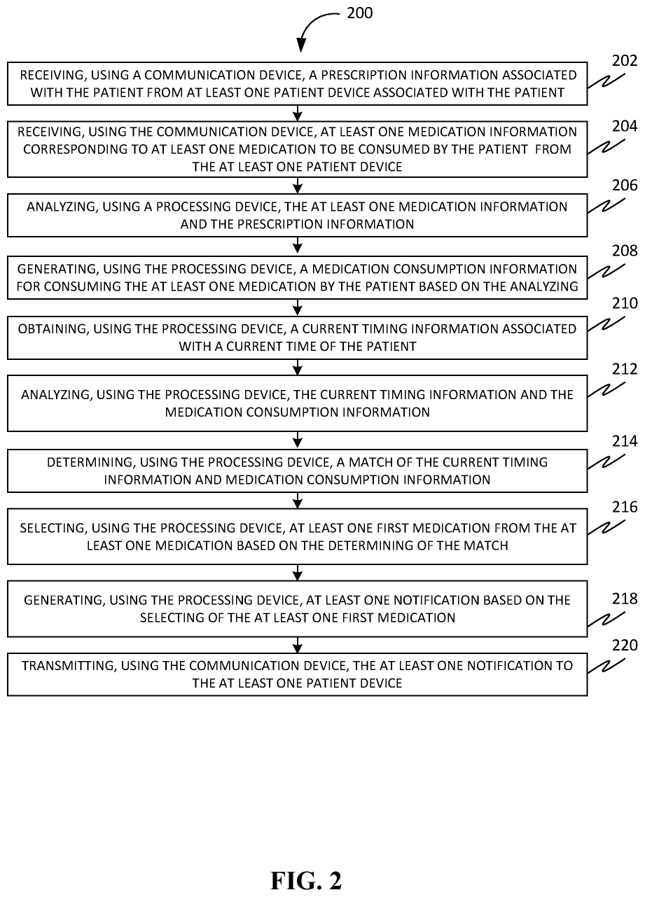

FIG. 2 is a flow chart of a method 200 of facilitating managing medication for a patient, in accordance with some embodiments. Accordingly, at 202, the method 200 may include receiving, using a communication device (such as a communication device 1002), a prescription information associated with the patient from at least one patient device (such as a at least one patient device 1104) associated with the patient. Further, the at least one patient device may include a smartphone, a tablet, a laptop, a smartwatch, etc. Further, the prescription information may include a patient name, a disease name, a medication name, a medication manufacturer company name, a medication dosage details, etc. Further, at 204, the method 200 may include receiving, using the communication device, at least one medication information corresponding to at least one medication to be consumed by the patient from the at least one patient device. Further, the at least one medication may include a medicine, a tablet, a syrup, an injectable medicine, etc. that may be consumed by the patient. Further, the at least one medication information may include a bar code and a QR code. Further, at 206, the method 200 may include analyzing, using a processing device (such as a processing device 1004), the at least one medication information and the prescription information. Further, at 208, the method 200 may include generating, using the processing device, a medication consumption information for consuming the at least one medication by the patient based on the analyzing. Further, the medication consumption information may include a medication name, medication time, medication frequency (week/days), etc. Further, the medication consumption information may include at least one medication identifier, at least one timing, and at least one dosage associated with the at least one medication. Further, at 210, the method 200 may include obtaining, using the processing device, a current timing information associated with a current time of the patient. Further, at 212, the method 200 may include analyzing, using the processing device, the current timing information and the medication consumption information. Further, at 214, the method 200 may include determining, using the processing device, a match of the current timing information and the medication consumption information. Further, at 216, the method 200 may include selecting, using the processing device, at least one first medication from the at least one medication based on the determining of the match. Further, at least one first medication may be to be consumed by the patient at the current time. Further, at 218, the method 200 may include generating, using the processing device, at least one notification based on the selecting of the at least one first medication. Further, the at least one notification may include at least one first medication consumption information from the medication consumption information. Further, the at least one first medication consumption information may be associated with the at least one first medication. Further, at 220, the method 200 may include transmitting, using the communication device, the at least one notification to the at least one patient device.

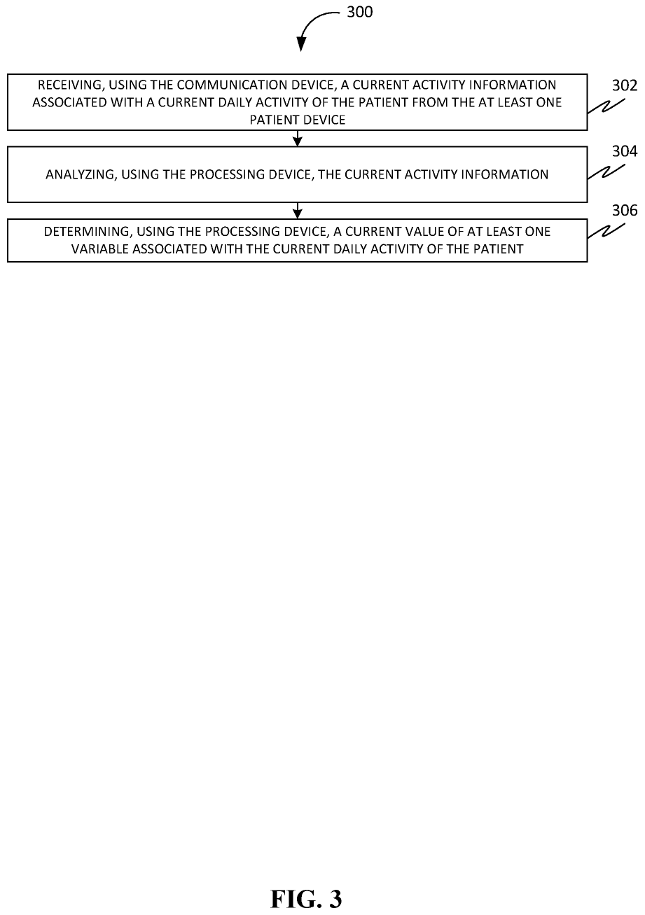

FIG. 3 is a flow chart of a method 300 of facilitating managing medication for the patient, in accordance with some embodiments. Accordingly, at 302, the method 300 may include receiving, using the communication device, a current activity information associated with a current daily activity of the patient from the at least one patient device. Further, the current activity information may include a sleeping time, a sleeping duration, a wake-up time, an activity duration, an activity starting time, an activity termination time, etc. Further, at 304, the method 300 may include analyzing, using the processing device, the current activity information. Further, at 306, the method 300 may include determining, using the processing device, a current value of at least one variable associated with the current daily activity of the patient. Further, the generating of the medication consumption information may be based on the determining of the current value of the at least one variable.

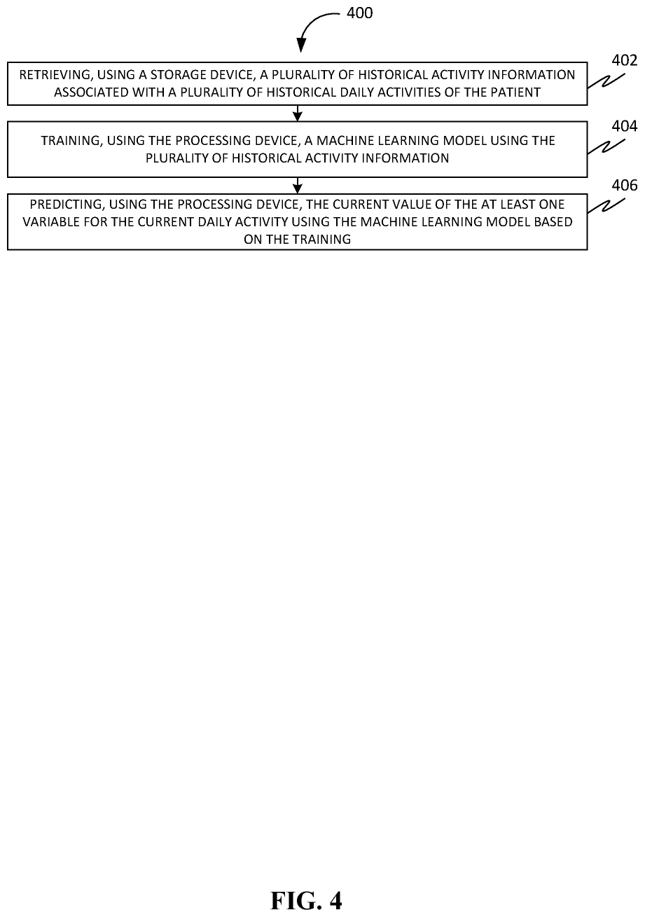

FIG. 4 is a flow chart of a method 400 of facilitating managing medication for the patient, in accordance with some embodiments. Accordingly, at 402, the method 400 may include retrieving, using a storage device, a plurality of historical activity information associated with a plurality of historical daily activities of the patient. Further, each of the plurality of historical activity information may be characterized by a historical value of the at least one variable associated with each of the plurality of historical daily activities. Further, at 404, the method 400 may include training, using the processing device, a machine learning model using the plurality of historical activity information. Further, the machine learning model may include a support vector machine. Further, the training of the machine learning model may include extracting at least one feature from the plurality of historical activity information. Further, the training of the machine learning model may include tuning at least one value of one or more parameters of the machine learning model based on the at least one feature. Further, at 406, the method 400 may include predicting, using the processing device, the current value of the at least one variable for the current daily activity using the machine learning model based on the training. Further, the determining of the current value of the at least one variable may be based on the predicting.

Further, in some embodiments, the at least one patient device may include at least one first sensor (such as at least one first sensor 1106). Further, the at least one first sensor may include a motion sensor, a camera, a blood pressure sensor, a heartbeat sensor, a gyroscope, an accelerometer, a thermal sensor, etc. Further, in some embodiments, the at least one first sensor may include a physiological sensor configured for detecting physiological changes in a body of the patient upon performing the current activity. Further, the at least one first sensor may be configured for generating each of the plurality of historical activity information by detecting a value of at least one activity characteristic of each of the plurality of historical daily activities performed by the patient. Further, the plurality of historical daily activities may include a plurality of successive daily activities performed successively by the patient in an order from the current time to at least one historical time.

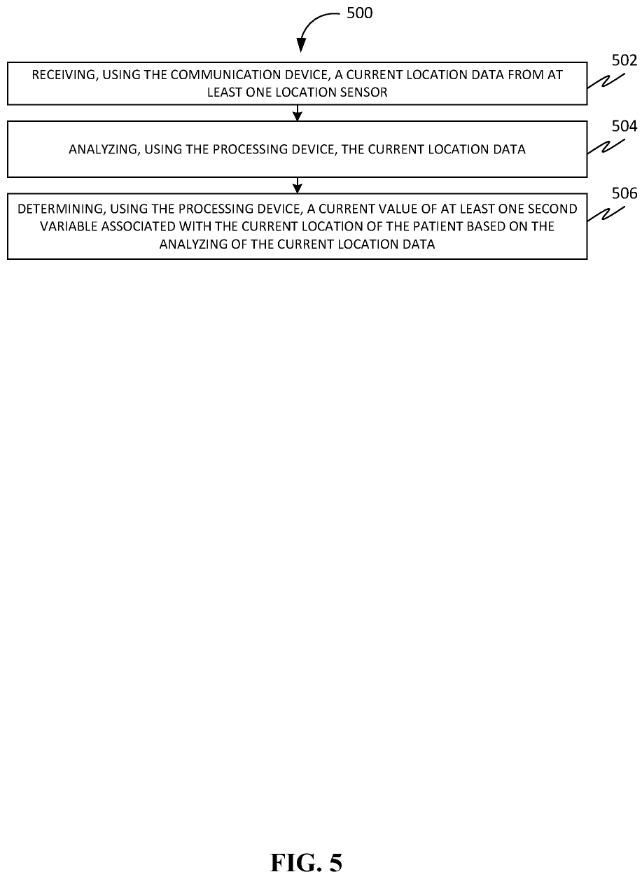

FIG. 5 is a flow chart of a method 500 of facilitating managing medication for the patient, in accordance with some embodiments. Accordingly, at 502, the method 500 may include receiving, using the communication device, a current location data from at least one location sensor (such as at least one location sensor 1202). Further, the at least one location sensor may be configured for generating the current location data based on detecting a current location associated with the patient. Further, the current location data may include location coordinates, a location name, etc. Further, at 504, the method 500 may include analyzing, using the processing device, the current location data. Further, at 506, the method 500 may include determining, using the processing device, a current value of at least one second variable associated with the current location of the patient based on the analyzing of the current location data. Further, the generating of the medication consumption information may be based on the determining of the current value of the at least one second variable.

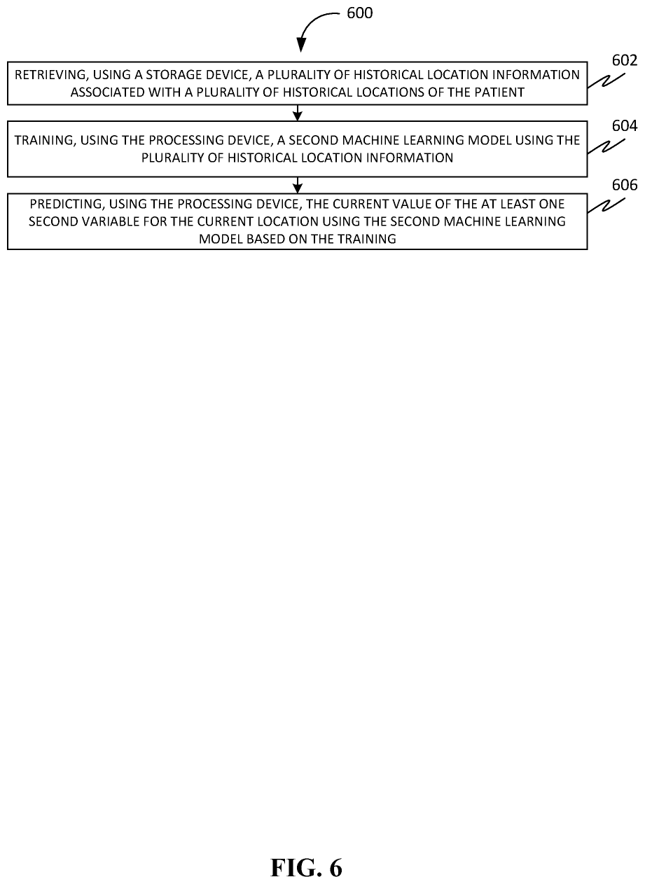

FIG. 6 is a flow chart of a method 600 of facilitating managing medication for the patient, in accordance with some embodiments. Accordingly, at 602, the method 600 may include retrieving, using a storage device, a plurality of historical location information associated with a plurality of historical locations of the patient. Further, each of the plurality of historical location information may be characterized by a historical value of the at least one second variable associated with each of the plurality of historical locations. Further, at 604, the method 600 may include training, using the processing device, a second machine learning model using the plurality of historical location information. Further, the second machine learning model may include a support vector machine. Further, the training of the second machine learning model may include extracting at least one second feature from the plurality of historical location information. Further, the training of the machine learning model may include tuning at least one value of one or more parameters of the second machine learning model based on the at least one second feature. Further, at 606, the method 600 may include predicting, using the processing device, the current value of the at least one second variable for the current location using the second machine learning model based on the training. Further, the determining of the current value of the at least one second variable may be based on the predicting.

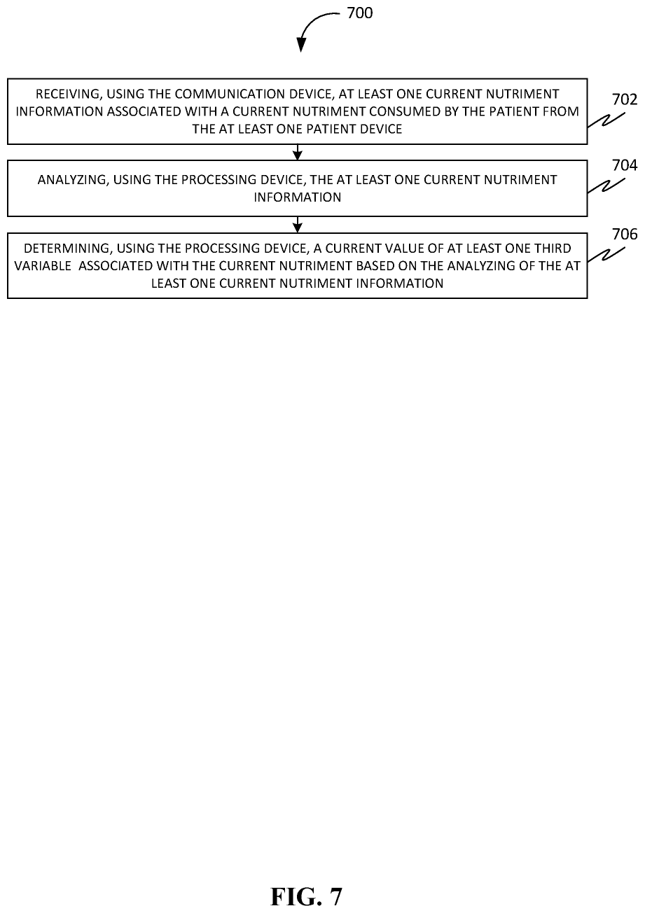

FIG. 7 is a flow chart of a method 700 of facilitating managing medication for the patient, in accordance with some embodiments. Accordingly, at 702, the method 700 may include receiving, using the communication device, at least one current nutriment information associated with a current nutriment consumed by the patient from the at least one patient device. Further, the current nutriment may include an edible item that may nourish a body of the patient. Further, in an instance, the current nutriment may include a food item, a beverage name, a food ingredient list, etc. Further, the at least one current nutriment information may include nutriment name, a nutriment amount, consumption time, temperature, nutriment manufacturer name, manufacturing or producing process, nutriment ingredients, etc. Further, at 704, the method 700 may include analyzing, using the processing device, the at least one current nutriment information. Further, at 706, the method 700 may include determining, using the processing device, a current value of at least one third variable associated with the current nutriment based on the analyzing of the at least one current nutriment information. Further, the generating of the medication consumption information may be based on the determining of the current value of the at least one third variable.

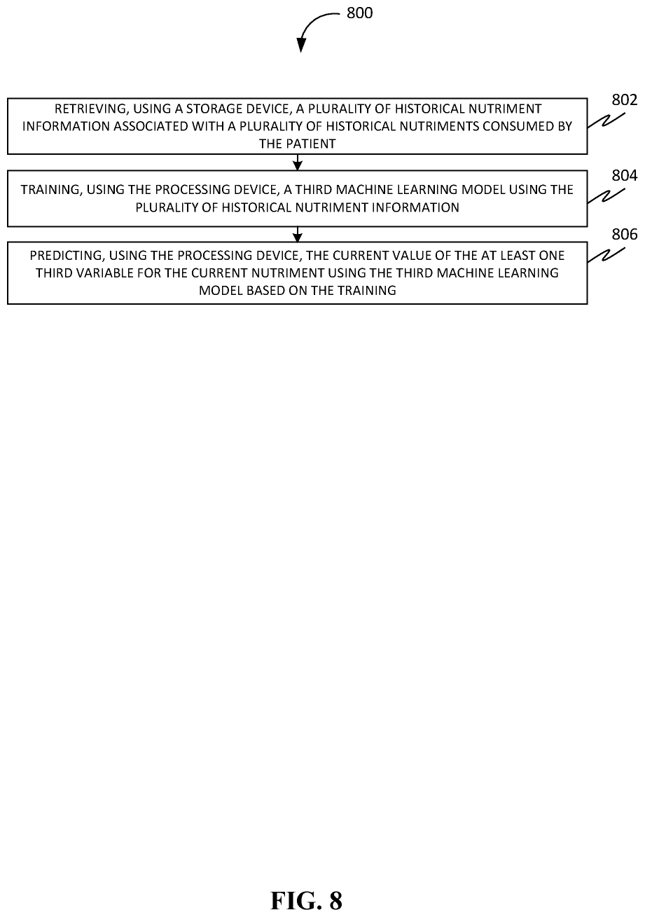

FIG. 8 is a flow chart of a method 800 of facilitating managing medication for the patient, in accordance with some embodiments. Accordingly, at 802, the method 800 may include retrieving, using a storage device, a plurality of historical nutriment information associated with a plurality of historical nutriments consumed by the patient. Further, each of the plurality of historical nutriment information may be characterized by a historical value of the at least one third variable associated with each of the plurality of historical nutriments. Further, at 804, the method 800 may include training, using the processing device, a third machine learning model using the plurality of historical nutriment information. Further, the third machine learning model may include a support vector machine. Further, the training of the third machine learning model may include extracting at least one third feature from the plurality of historical nutriment information. Further, the training of the machine learning model may include tuning at least one value of one or more parameters of the third machine learning model based on the at least one third feature. Further, at 806, the method 800 may include predicting, using the processing device, the current value of the at least one third variable for the current nutriment using the third machine learning model based on the training. Further, the determining of the current value of the at least one third variable may be based on the predicting.

Further, in some embodiments, the at least one patient device may include at least one second sensor (such as at least one second sensor 1302). Further, the at least one second sensor may include a physiological sensor, a blood sugar sensor, an emotional sensor, a heartbeat sensor, a blood pressure sensor, a camera, an audio sensor, etc. Further, the at least one second sensor may be configured for generating each of the plurality of historical nutriment information by detecting a value of at least one nutriment characteristic of each of the plurality of historical nutriments consumed by the patient. Further, the plurality of historical nutriments may include a plurality of successive nutriments consumed successively by the patient in an order from the current time to at least one historical time.

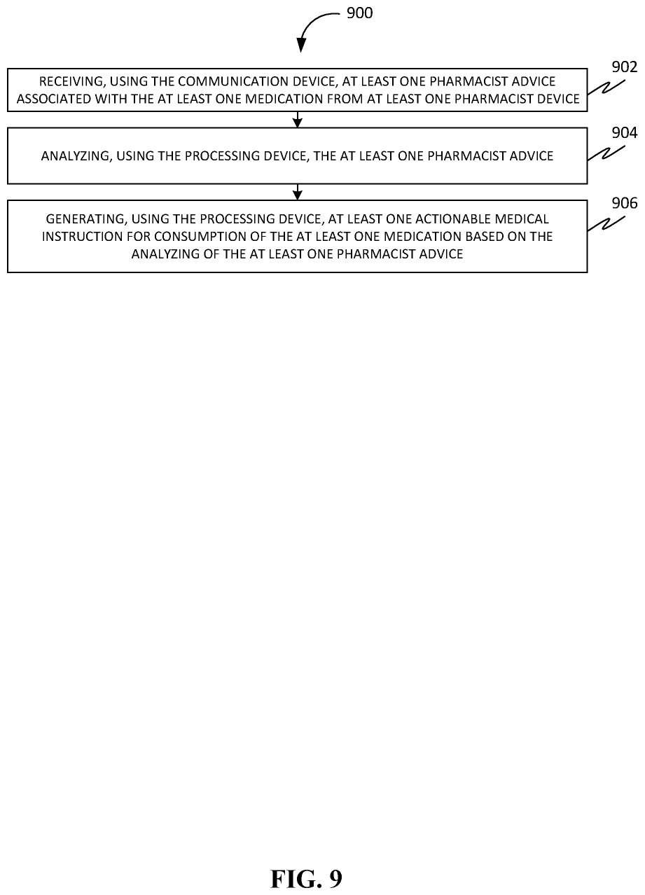

FIG. 9 is a flow chart of a method 900 of facilitating managing medication for the patient, in accordance with some embodiments. Further, at 902, the method 900 may include receiving, using the communication device, at least one pharmacist advice associated with the at least one medication from at least one pharmacist device (such as at least one pharmacist device 1402). Further, the at least one pharmacist device may include a laptop, a mobile, a smartphone, etc. Further, the at least one pharmacist advice may include directions on how to consume the at least one medication, a storage temperature and a storage place for storing the at least one medication, etc. Further, at 904, the method 900 may include analyzing, using the processing device, the at least one pharmacist advice. Further, at 906, the method 900 may include generating, using the processing device, at least one actionable medical instruction for consumption of the at least one medication based on the analyzing of the at least one pharmacist advice. Further, the generating of the medical consumption information may be based on the at least one actionable medical instruction.

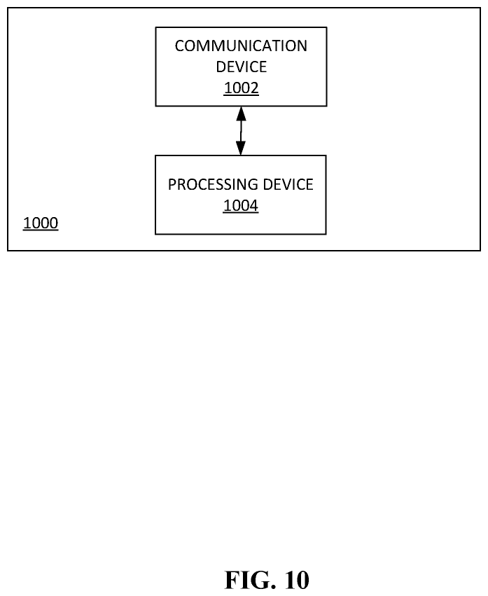

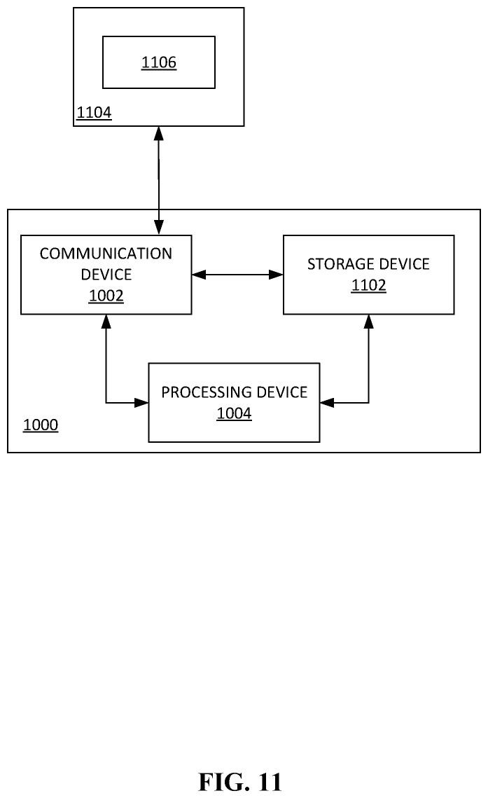

FIG. 10 is a block diagram of a system 1000 of facilitating managing medication for a patient, in accordance with some embodiments. Accordingly, the system 1000 may include a communication device 1002 configured for receiving a prescription information associated with the patient from at least one patient device 1104 (as shown in FIG. 11) associated with the patient. Further, the communication device 1002 may be configured for receiving at least one medication information corresponding to at least one medication to be consumed by the patient from the at least one patient device 1104. Further, the communication device 1002 may be configured for transmitting at least one notification to the at least one patient device 1104.

Further, the system 1000 may include a processing device 1004 communicatively coupled with the communication device 1002. Further, the processing device 1004 may be configured for analyzing the at least one medication information and the prescription information. Further, the processing device 1004 may be configured for generating a medication consumption information for consuming the at least one medication by the patient based on the analyzing. Further, the medication consumption information may include at least one medication identifier, at least one timing, and at least one dosage associated with the at least one medication. Further, the processing device 1004 may be configured for obtaining a current timing information associated with a current time of the patient. Further, the processing device 1004 may be configured for analyzing the current timing information and the medication consumption information. Further, the processing device 1004 may be configured for determining a match of the current timing information and the medication consumption information. Further, the processing device 1004 may be configured for selecting at least one first medication from the at least one medication based on the determining of the match. Further, at least one first medication may be to be consumed by the patient at the current time. Further, the processing device 1004 may be configured for generating the at least one notification based on the selecting of the at least one first medication. Further, the at least one notification may include at least one first medication consumption information from the medication consumption information. Further, the at least one first medication consumption information may be associated with the at least one first medication.

Further, in some embodiments, the communication device 1002 may be configured for receiving a current activity information associated with a current daily activity of the patient from the at least one patient device 1104. Further, the processing device 1004 may be configured for analyzing the current activity information. Further, the processing device 1004 may be configured for determining a current value of at least one variable associated with the current daily activity of the patient. Further, the generating of the medication consumption information may be based on the determining of the current value of the at least one variable.

In further embodiments, the system 1000 may include a storage device 1102 (as shown in FIG. 11) communicatively coupled with the processing device 1004. Further, the storage device 1102 may be configured for retrieving a plurality of historical activity information associated with a plurality of historical daily activities of the patient. Further, each of the plurality of historical activity information may be characterized by a historical value of the at least one variable associated with each of the plurality of historical daily activities. Further, the processing device 1004 may be configured for training a machine learning model using the plurality of historical activity information. Further, the machine learning model may include a support vector machine. Further, the training of the machine learning model may include extracting at least one feature from the plurality of historical activity information. Further, the training of the machine learning model may include tuning at least one value of one or more parameters of the machine learning model based on the at least one feature. Further, the processing device 1004 may be configured for predicting the current value of the at least one variable for the current daily activity using the machine learning model based on the training. Further, the determining of the current value of the at least one variable may be based on the predicting.

Further, in some embodiments, the at least one patient device 1104 may include at least one first sensor 1106 (as shown in FIG. 11). Further, the at least one first sensor 1106 may be configured for generating each of the plurality of historical activity information by detecting a value of at least one activity characteristic of each of the plurality of historical daily activities performed by the patient. Further, the plurality of historical daily activities may include a plurality of successive daily activities performed successively by the patient in an order from the current time to at least one historical time.

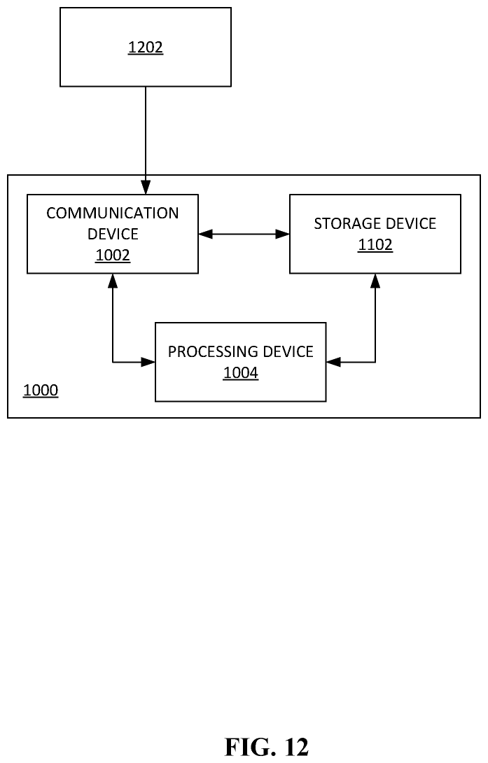

Further, in some embodiments, the communication device 1002 may be configured for receiving a current location data from at least one location sensor 1202 (as shown in FIG. 12). Further, the at least one location sensor 1202 may be configured for generating the current location data based on detecting a current location associated with the patient. Further, the processing device 1004 may be configured for analyzing the current location data. Further, the processing device 1004 may be configured for determining a current value of at least one second variable associated with the current location of the patient based on the analyzing of the current location data. Further, the generating of the medication consumption information may be based on the determining of the current value of the at least one second variable.

In further embodiments, the storage device 1102 may be configured for retrieving a plurality of historical location information associated with a plurality of historical locations of the patient. Further, each of the plurality of historical location information may be characterized by a historical value of the at least one second variable associated with each of the plurality of historical locations. Further, the processing device 1004 may be configured for training a second machine learning model using the plurality of historical location information. Further, the second machine learning model may include a support vector machine. Further, the training of the second machine learning model may include extracting at least one second feature from the plurality of historical location information. Further, the training of the second machine learning model may include tuning at least one value of one or more parameters of the second machine learning model based on the at least one second feature. Further, the processing device 1004 may be configured for predicting the current value of the at least one second variable for the current location using the second machine learning model based on the training. Further, the determining of the current value of the at least one second variable may be based on the predicting.

Further, in some embodiments, the communication device 1002 may be configured for receiving at least one current nutriment information associated with a current nutriment consumed by the patient from the at least one patient device 1104. Further, the processing device 1004 may be configured for analyzing the at least one current nutriment information. Further, the processing device 1004 may be configured for determining a current value of at least one third variable associated with the current nutriment based on the analyzing of the at least one current nutriment information. Further, the generating of the medication consumption information may be based on the determining of the current value of the at least one third variable.

In further embodiments, the storage device 1102 may be configured for retrieving a plurality of historical nutriment information associated with a plurality of historical nutriments consumed by the patient. Further, each of the plurality of historical nutriment information may be characterized by a historical value of the at least one third variable associated with each of the plurality of historical nutriments. Further, the processing device 1004 may be configured for training a third machine learning model using the plurality of historical nutriment information. Further, the third machine learning model may include a support vector machine. Further, the training of the third machine learning model may include extracting at least one third feature from the plurality of historical nutriment information. Further, the training of the second machine learning model may include tuning at least one value of one or more parameters of the third machine learning model based on the at least one third feature. Further, the processing device 1004 may be configured for predicting the current value of the at least one third variable for the current nutriment using the third machine learning model based on the training. Further, the determining of the current value of the at least one third variable may be based on the predicting.

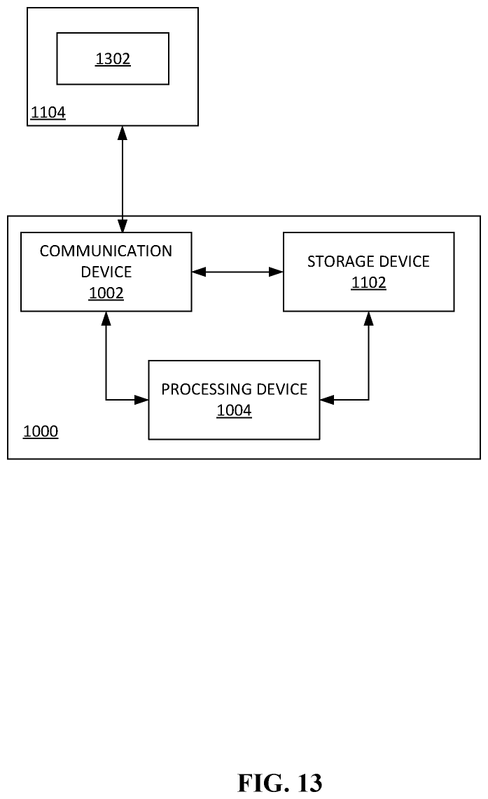

Further, in some embodiments, the at least one patient device 1104 may include at least one second sensor 1302 (as shown in FIG. 13). Further, the at least one second sensor 1302 may be configured for generating each of the plurality of historical nutriment information by detecting a value of at least one nutriment characteristic of each of the plurality of historical nutriments consumed by the patient. Further, the plurality of historical nutriments may include a plurality of successive nutriments consumed successively by the patient in an order from the current time to at least one historical time.

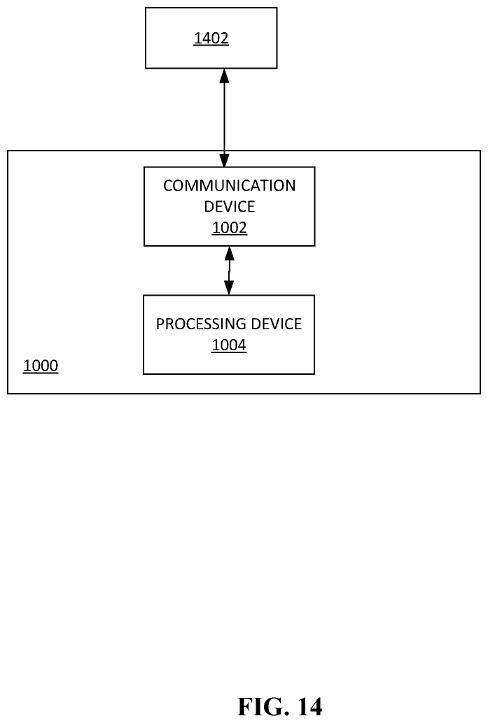

Further, in some embodiments, the communication device 1002 may be configured for receiving at least one pharmacist advice associated with the at least one medication from at least one pharmacist device 1402 (as shown in FIG. 14). Further, the processing device 1004 may be configured for analyzing the at least one pharmacist advice 1402. Further, the processing device 1004 may be configured for generating at least one actionable medical instruction for consumption of the at least one medication based on the analyzing of the at least one pharmacist advice. Further, the generating of the medical consumption information may be based on the at least one actionable medical instruction.

FIG. 11 is a block diagram of the system 1000 of facilitating managing medication for the patient, in accordance with further embodiments.

FIG. 12 is a block diagram of the system 1000 of facilitating managing medication for the patient, in accordance with further embodiments.

FIG. 13 is a block diagram of the system 1000 of facilitating managing medication for the patient, in accordance with further embodiments.

FIG. 14 is a block diagram of the system 1000 of facilitating managing medication for the patient, in accordance with further embodiments.

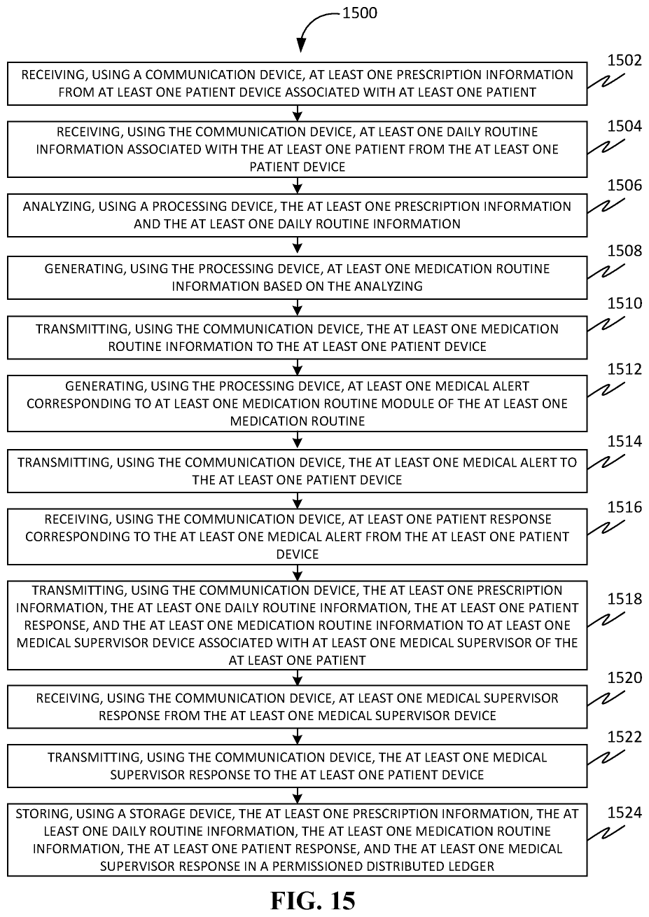

FIG. 15 is a flowchart of a method 1500 for facilitating medical assistance to patients, in accordance with some embodiments.

Further, at 1502, the method 1500 may include a step of receiving, using a communication device, at least one prescription information from at least one patient device associated with at least one patient. Further, the at least one prescription information may include information associated with at least one medication prescribed to the at least one patient.

Further, at 1504, the method 1500 may include a step of receiving, using the communication device, at least one daily routine information associated with the at least one patient from the at least one patient device. Further, the at least one daily routine information may reflect a daily routine of the at least one patient.

Further, at 1506, the method 1500 may include a step of analyzing, using a processing device, the at least one prescription information and the at least one daily routine information.

Further, at 1508, the method 1500 may include a step of generating, using the processing device, at least one medication routine information based on the analyzing. Further, the at least one medication routine information may reflect at least one medication routine the at least one patient may follow.

Further, at 1510, the method 1500 may include a step of transmitting, using the communication device, the at least one medication routine information to the at least one patient device.

Further, at 1512, the method 1500 may include a step of generating, using the processing device, at least one medical alert corresponding to at least one medication routine module of the at least one medication routine.

Further, at 1514, the method 1500 may include a step of transmitting, using the communication device, the at least one medical alert to the at least one patient device. Further, the at least one patient device may include but may not be limited to a smartphone, a smart band, a smartwatch, a smart speaker, a smart display, etc.

Further, at 1516, the method 1500 may include a step of receiving, using the communication device, at least one patient response corresponding to the at least one medical alert from the at least one patient device.

Further, at 1518, the method 1500 may include a step of transmitting, using the communication device, the at least one prescription information, the at least one daily routine information, the at least one patient response, and the at least one medication routine information to at least one medical supervisor device associated with at least one medical supervisor of the at least one patient.

Further, at 1520, the method 1500 may include a step of receiving, using the communication device, at least one medical supervisor response from the at least one medical supervisor device.

Further, at 1522, the method 1500 may include a step of transmitting, using the communication device, the at least one medical supervisor response to the at least one patient device.

Further, at 1524, the method 1500 may include a step of storing, using a storage device, the at least one prescription information, the at least one daily routine information, the at least one medication routine information, the at least one patient response, and the at least one medical supervisor response in a permissioned distributed ledger.

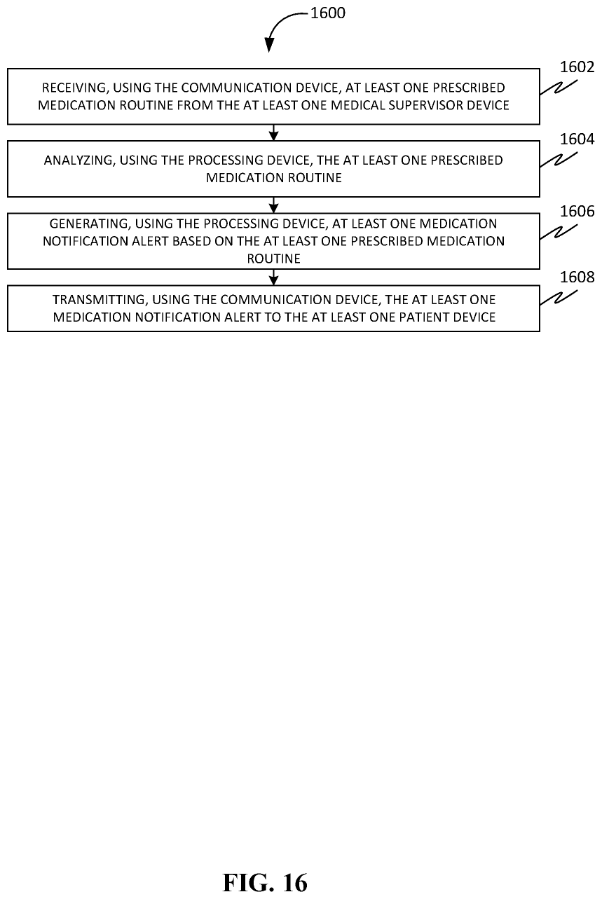

FIG. 16 is a flowchart of a method 1600 for facilitating medical assistance to patients, in accordance with some embodiments.

Further, at 1602, the method 1600 may include a step of receiving, using the communication device, at least one prescribed medication routine from the at least one medical supervisor device.

Further, at 1604, the method 1600 may include a step of analyzing, using the processing device, the at least one prescribed medication routine.

Further, at 1606, the method 1600 may include a step of generating, using the processing device, at least one medication notification alert based on the at least one prescribed medication routine.

Further, at 1608, the method 1600 may include a step of transmitting, using the communication device, the at least one medication notification alert to the at least one patient device.

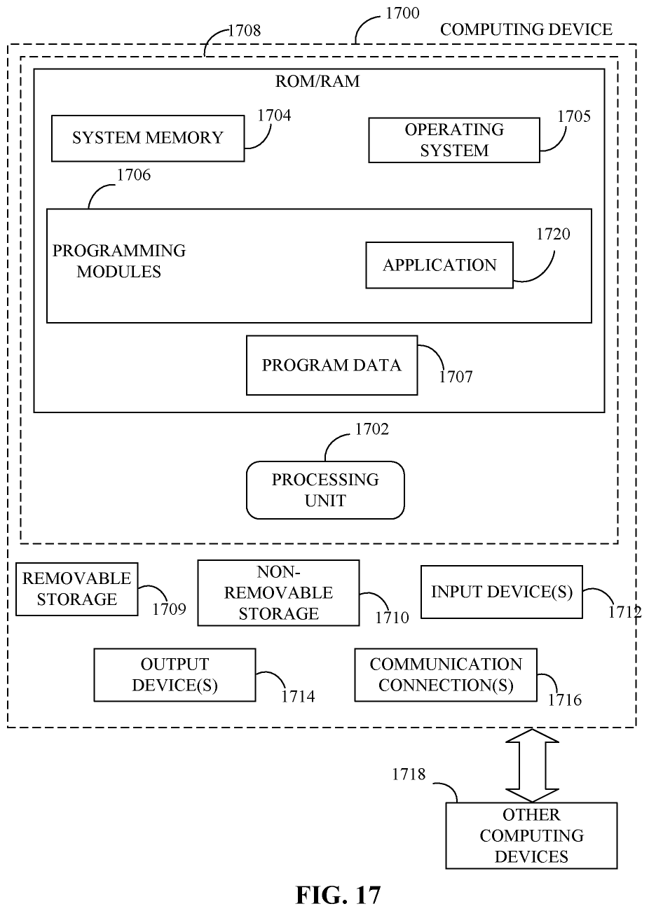

With reference to FIG. 17, a system consistent with an embodiment of the disclosure may include a computing device or cloud service, such as computing device 1700. In a basic configuration, computing device 1700 may include at least one processing unit 1702 and a system memory 1704. Depending on the configuration and type of computing device, system memory 1704 may comprise, but is not limited to, volatile (e.g. random-access memory (RAM)), non-volatile (e.g. read-only memory (ROM)), flash memory, or any combination. System memory 1704 may include operating system 1705, one or more programming modules 1706, and may include a program data 1707. Operating system 1705, for example, may be suitable for controlling computing device 1700′s operation. In one embodiment, programming modules 1706 may include image-processing module, machine learning module. Furthermore, embodiments of the disclosure may be practiced in conjunction with a graphics library, other operating systems, or any other application program and is not limited to any particular application or system. This basic configuration is illustrated in FIG. 17 by those components within a dashed line 1708.

Computing device 1700 may have additional features or functionality. For example, computing device 1700 may also include additional data storage devices (removable and/or non-removable) such as, for example, magnetic disks, optical disks, or tape. Such additional storage is illustrated in FIG. 17 by a removable storage 1709 and a non-removable storage 1710. Computer storage media may include volatile and non-volatile, removable and non-removable media implemented in any method or technology for storage of information, such as computer-readable instructions, data structures, program modules, or other data. System memory 1704, removable storage 1709, and non-removable storage 1710 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1700. Any such computer storage media may be part of device 1700. Computing device 1700 may also have input device(s) 1712 such as a keyboard, a mouse, a pen, a sound input device, a touch input device, a location sensor, a camera, a biometric sensor, etc. Output device(s) 1714 such as a display, speakers, a printer, etc. may also be included. The aforementioned devices are examples and others may be used.

Computing device 1700 may also contain a communication connection 1716 that may allow device 1700 to communicate with other computing devices 1718, such as over a network in a distributed computing environment, for example, an intranet or the Internet. Communication connection 1716 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1704, including operating system 1705. While executing on processing unit 1702, programming modules 1706 (e.g., application 1720 such as a media player) may perform processes including, for example, one or more stages of methods, algorithms, systems, applications, servers, databases as described above. The aforementioned process is an example, and processing unit 1702 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. A method of facilitating managing medication for a patient, the method comprising:
receiving, using a communication device, a prescription information associated with the patient from at least one patient device associated with the patient;
receiving, using the communication device, at least one medication information corresponding to at least one medication to be consumed by the patient from the at least one patient device;
analyzing, using a processing device, the at least one medication information and the prescription information;
generating, using the processing device, a medication consumption information for consuming the at least one medication by the patient based on the analyzing, wherein the medication consumption information comprises at least one medication identifier, at least one timing, and at least one dosage associated with the at least one medication;
obtaining, using the processing device, a current timing information associated with a current time of the patient;
analyzing, using the processing device, the current timing information and the medication consumption information;
determining, using the processing device, a match of the current timing information and the medication consumption information;
selecting, using the processing device, at least one first medication from the at least one medication based on the determining of the match, wherein at least one first medication is to be consumed by the patient at the current time;
generating, using the processing device, at least one notification based on the selecting of the at least one first medication, wherein the at least one notification comprises at least one first medication consumption information from the medication consumption information, wherein the at least one first medication consumption information is associated with the at least one first medication; and
transmitting, using the communication device, the at least one notification to the at least one patient device.

2. The method of claim 1 further comprising:
receiving, using the communication device, a current activity information associated with a current daily activity of the patient from the at least one patient device;
analyzing, using the processing device, the current activity information; and
determining, using the processing device, a current value of at least one variable associated with the current daily activity of the patient, wherein the generating of the medication consumption information is further based on the determining of the current value of the at least one variable.

3. The method of claim 2 further comprising:
retrieving, using a storage device, a plurality of historical activity information associated with a plurality of historical daily activities of the patient, wherein each of the plurality of historical activity information is characterized by a historical value of the at least one variable associated with each of the plurality of historical daily activities;
training, using the processing device, a machine learning model using the plurality of historical activity information, wherein the machine learning model comprises a support vector machine, wherein the training of the machine learning model comprises:
extracting at least one feature from the plurality of historical activity information; and
tuning at least one value of one or more parameters of the machine learning model based on the at least one feature; and
predicting, using the processing device, the current value of the at least one variable for the current daily activity using the machine learning model based on the training, wherein the determining of the current value of the at least one variable is further based on the predicting.

4. The method of claim 3, wherein the at least one patient device comprises at least one first sensor, wherein the at least one first sensor is configured for generating each of the plurality of historical activity information by detecting a value of at least one activity characteristic of each of the plurality of historical daily activities performed by the patient, wherein the plurality of historical daily activities comprises a plurality of successive daily activities performed successively by the patient in an order from the current time to at least one historical time.

5. The method of claim 1 further comprising:
receiving, using the communication device, a current location data from at least one location sensor, wherein the at least one location sensor is configured for generating the current location data based on detecting a current location associated with the patient;
analyzing, using the processing device, the current location data; and
determining, using the processing device, a current value of at least one second variable associated with the current location of the patient based on the analyzing of the current location data, wherein the generating of the medication consumption information is further based on the determining of the current value of the at least one second variable.

6. The method of claim 5 further comprising:
retrieving, using a storage device, a plurality of historical location information associated with a plurality of historical locations of the patient, wherein each of the plurality of historical location information is characterized by a historical value of the at least one second variable associated with each of the plurality of historical locations;
training, using the processing device, a second machine learning model using the plurality of historical location information, wherein the second machine learning model comprises a support vector machine, wherein the training of the second machine learning model comprises:
extracting at least one second feature from the plurality of historical location information; and
tuning at least one value of one or more parameters of the second machine learning model based on the at least one second feature; and
predicting, using the processing device, the current value of the at least one second variable for the current location using the second machine learning model based on the training, wherein the determining of the current value of the at least one second variable is further based on the predicting.

7. The method of claim 1 further comprising:
receiving, using the communication device, at least one current nutriment information associated with a current nutriment consumed by the patient from the at least one patient device;
analyzing, using the processing device, the at least one current nutriment information; and
determining, using the processing device, a current value of at least one third variable associated with the current nutriment based on the analyzing of the at least one current nutriment information, wherein the generating of the medication consumption information is further based on the determining of the current value of the at least one third variable.

8. The method of claim 7 further comprising:
retrieving, using a storage device, a plurality of historical nutriment information associated with a plurality of historical nutriments consumed by the patient, wherein each of the plurality of historical nutriment information is characterized by a historical value of the at least one third variable associated with each of the plurality of historical nutriments;
training, using the processing device, a third machine learning model using the plurality of historical nutriment information, wherein the third machine learning model comprises a support vector machine, wherein the training of the third machine learning model comprises:
extracting at least one third feature from the plurality of historical nutriment information; and
tuning at least one value of one or more parameters of the third machine learning model based on the at least one third feature; and
predicting, using the processing device, the current value of the at least one third variable for the current nutriment using the third machine learning model based on the training, wherein the determining of the current value of the at least one third variable is further based on the predicting.

9. The method of claim 8, wherein the at least one patient device comprises at least one second sensor, wherein the at least one second sensor is configured for generating each of the plurality of historical nutriment information by detecting a value of at least one nutriment characteristic of each of the plurality of historical nutriments consumed by the patient, wherein the plurality of historical nutriments comprises a plurality of successive nutriments consumed successively by the patient in an order from the current time to at least one historical time.

10. The method of claim 1 further comprising:
receiving, using the communication device, at least one pharmacist advice associated with the at least one medication from at least one pharmacist device;
analyzing, using the processing device, the at least one pharmacist advice; and
generating, using the processing device, at least one actionable medical instruction for consumption of the at least one medication based on the analyzing of the at least one pharmacist advice, wherein the generating of the medical consumption information is further based on the at least one actionable medical instruction.

11. A system of facilitating managing medication for a patient, the system comprising:
a communication device configured for:
receiving a prescription information associated with the patient from at least one patient device associated with the patient;
receiving at least one medication information corresponding to at least one medication to be consumed by the patient from the at least one patient device; and
transmitting at least one notification to the at least one patient device; and
a processing device communicatively coupled with the communication device, wherein the processing device is configured for:
analyzing the at least one medication information and the prescription information;
generating a medication consumption information for consuming the at least one medication by the patient based on the analyzing, wherein the medication consumption information comprises at least one medication identifier, at least one timing, and at least one dosage associated with the at least one medication;
obtaining a current timing information associated with a current time of the patient;
analyzing the current timing information and the medication consumption information;
determining a match of the current timing information and the medication consumption information;
selecting at least one first medication from the at least one medication based on the determining of the match, wherein at least one first medication is to be consumed by the patient at the current time; and
generating the at least one notification based on the selecting of the at least one first medication, wherein the at least one notification comprises at least one first medication consumption information from the medication consumption information, wherein the at least one first medication consumption information is associated with the at least one first medication.

12. The system of claim 11, wherein the communication device is further configured for receiving a current activity information associated with a current daily activity of the patient from the at least one patient device, wherein the processing device is configured for:
analyzing the current activity information; and
determining a current value of at least one variable associated with the current daily activity of the patient, wherein the generating of the medication consumption information is further based on the determining of the current value of the at least one variable.

13. The system of claim 12 further comprising a storage device communicatively coupled with the processing device, wherein the storage device is configured for retrieving a plurality of historical activity information associated with a plurality of historical daily activities of the patient, wherein each of the plurality of historical activity information is characterized by a historical value of the at least one variable associated with each of the plurality of historical daily activities, wherein the processing device is configured for:
training a machine learning model using the plurality of historical activity information, wherein the machine learning model comprises a support vector machine, wherein the training of the machine learning model comprises:
extracting at least one feature from the plurality of historical activity information; and
tuning at least one value of one or more parameters of the machine learning model based on the at least one feature; and
predicting the current value of the at least one variable for the current daily activity using the machine learning model based on the training, wherein the determining of the current value of the at least one variable is further based on the predicting.

14. The system of claim 13, wherein the at least one patient device comprises at least one first sensor, wherein the at least one first sensor is configured for generating each of the plurality of historical activity information by detecting a value of at least one activity characteristic of each of the plurality of historical daily activities performed by the patient, wherein the plurality of historical daily activities comprises a plurality of successive daily activities performed successively by the patient in an order from the current time to at least one historical time.

15. The system of claim 11, wherein the communication device is configured for receiving a current location data from at least one location sensor, wherein the at least one location sensor is configured for generating the current location data based on detecting a current location associated with the patient, wherein the processing device is configured for:
analyzing the current location data; and
determining a current value of at least one second variable associated with the current location of the patient based on the analyzing of the current location data, wherein the generating of the medication consumption information is further based on the determining of the current value of the at least one second variable.

16. The system of claim 15 further comprising a storage device communicatively coupled with the processing device, wherein the storage device is configured for retrieving a plurality of historical location information associated with a plurality of historical locations of the patient, wherein each of the plurality of historical location information is characterized by a historical value of the at least one second variable associated with each of the plurality of historical locations, wherein the processing device is configured for:
training a second machine learning model using the plurality of historical location information, wherein the second machine learning model comprises a support vector machine, wherein the training of the second machine learning model comprises:
extracting at least one second feature from the plurality of historical location information; and
tuning at least one value of one or more parameters of the second machine learning model based on the at least one second feature; and
predicting the current value of the at least one second variable for the current location using the second machine learning model based on the training, wherein the determining of the current value of the at least one second variable is further based on the predicting.

17. The system of claim 11, wherein the communication device is configured for receiving at least one current nutriment information associated with a current nutriment consumed by the patient from the at least one patient device, wherein the processing device is configured for:
analyzing the at least one current nutriment information; and
determining a current value of at least one third variable associated with the current nutriment based on the analyzing of the at least one current nutriment information, wherein the generating of the medication consumption information is further based on the determining of the current value of the at least one third variable.

18. The system of claim 17 further comprising a storage device communicatively coupled with the processing device, wherein the storage device is configured for retrieving a plurality of historical nutriment information associated with a plurality of historical nutriments consumed by the patient, wherein each of the plurality of historical nutriment information is characterized by a historical value of the at least one third variable associated with each of the plurality of historical nutriments, wherein the processing device is configured for:
training a third machine learning model using the plurality of historical nutriment information, wherein the third machine learning model comprises a support vector machine, wherein the training of the third machine learning model comprises:
extracting at least one third feature from the plurality of historical nutriment information; and
tuning at least one value of one or more parameters of the third machine learning model based on the at least one third feature; and
predicting the current value of the at least one third variable for the current nutriment using the third machine learning model based on the training, wherein the determining of the current value of the at least one third variable is further based on the predicting.

19. The system of claim 18, wherein the at least one patient device comprises at least one second sensor, wherein the at least one second sensor is configured for generating each of the plurality of historical nutriment information by detecting a value of at least one nutriment characteristic of each of the plurality of historical nutriments consumed by the patient, wherein the plurality of historical nutriments comprises a plurality of successive nutriments consumed successively by the patient in an order from the current time to at least one historical time.

20. The system of claim 11, wherein the communication device is configured for receiving at least one pharmacist advice associated with the at least one medication from at least one pharmacist device, wherein the processing device is configured for:
analyzing the at least one pharmacist advice; and
generating at least one actionable medical instruction for consumption of the at least one medication based on the analyzing of the at least one pharmacist advice, wherein the generating of the medical consumption information is further based on the at least one actionable medical instruction.
